(19) United States
(12) Patent Application Publication
(10) Pub. No.: US 2014/0326240 A1
(43) Pub. Date: Nov. 6, 2014

(54) Warming Vapor Pad

(71) Applicant: Rita A. Debski, Freehold, NJ (US)

(72) Inventor: Rita A. Debski, Freehold, NJ (US)

(21) Appl. No.: 13/874,553

(22) Filed: May 1, 2013

(51) Int. Cl. A61M 15/00

(52) U.S. Cl. CPC A61M 15/00
USPC 128/203.26

(57) ABSTRACT

A warming vapor pad to treat respiratory syncytial virus infections in babies, children and adults includes a fabric impregnated with a topical ointment providing medicated vapors to enhance nasal inhalation, with the fabric being superimposed on an elastic thermal wrap for application as a single integrated unit placed on a sitting or reclining user's chest.

### CROSS-REFERENCE TO RELATED APPLICATIONS

None

### STATEMENT REGARDING FEDERALLY SPONSORED RESEARCH OR DEVELOPMENT

Research and development of this invention and Application have not been federally sponsored, and no rights are given under any Federal program.

### REFERENCE TO A MICROFICHE APPENDIX

Not applicable.

### BACKGROUND OF THE INVENTION

1. Field of the Invention

This invention relates to respiratory syncytial virus infection (RSV), in general, and to an improved management and treatment technique for RSV suffering babies and infant children, in particular.

2. Description of the Related Art

As is understood in the medical profession, respiratory syncytial virus infection attacks one's nose, eyes, throat and lungs as to which the body never becomes immune. As is also understood, infants develop more serious problems, like wheezing or trouble breathing. Babies and infants younger than 6 months, especially those born prematurely, often display serious symptoms which can lead to such other problems as pneumonia. As is additionally understood, pursuant to recommendations of the American Academy of Pediatrics, most physicians choose not to recommend any medications for children under 4 years of age, which makes the infection far more disheartening for those parents thus unable to treat their child at home.

By and large, management and treatment of patients with all degrees of this RSV severity is accepted as necessary to relieve symptoms and reduce the likelihood of long term illnesses caused by the virus. For mild cases of RSV, the treatment is typically focused on relieving the symptoms—cough and cold medicines (which vary in efficacy) and bronchodilators such as metaproterenol or albuterol have been employed to help relieve whatever chest congestion and wheezing develops. Additional studies have assessed the effectiveness of these bronchodilators to suggest that epinephrine, racemic epinephrine, and ipratropium bromide may have a clinical role in the management of what acute bronchiolitis may develop. However, none of these treatments are readily available to babies, infants, and children under the age of 4 years.

As will be appreciated, the basic premise for managing mild to moderate RSV infection at home is to control the symptoms and make the patient comfortable. Thus, air temperatures are sought to be maintained between 70° F. and 72° F. so that the air is not overly, dry, nasal and nasal secretions are vigorously and frequently suctioned to keep the nasal airway clear and to reduce infant fatigue due to attempting to breathe through a mucous obstruction, and fluid levels are to be maintained to avoid dehydration. If the symptoms, however, become more pronounced, or if a child suffering these conditions seems particularly ill, visits to the pediatrician are very common. There, a physician can assess the severity of the disease to determine if hospitalization is required.

Where such severe RSV symptoms are diagnosed, several possible hospital treatments might follow: a) vigorous suctioning of nasal secretions; b) intravenous maintaining of fluid levels to combat dehydration; c) administering humidified oxygen via mask, tent or nasal tubing to maintain oxygen saturation of arterial blood at or greater than 95%; and d) providing mechanical ventilation for the child with an impending respiratory failure.

As will further be understood, administering ribavirin by aerosol is a medically accepted manner of treating severe RSV disease in infants and children. Being a broad-spectrum antiviral with virustatic effects, such ribavirin inhalation solutions serve to inhibit RSV replication during the active replication phase—while at the same time, significantly improving arterial blood oxygenation, decreasing illness severity and decreasing viral shedding. Because such antiviral agent is confined strictly in a hospital setting where it is delivered via a small particle aerosol generator whose operation is undertaken only by physicians and support staff familiar with the specific ventilator being used and the mode of administration of the drug, treating RSV of an infant or child at home this way is not a management technique for a parent to oversee. This becomes all the more so as this antiviral agent is delivered to the child or infant through an oxygen hood, oxygen tent or face mask attached to the generator, and as the ribavirin is not to be administered with any other aerosol generating device, or together with other aerosolized medications. As such, its use is wholly restricted to the hospital environment.

### OBJECTS OF THE INVENTION

It is an object of the invention, therefore, to provide some new and improved management and treatment technique for RSV suffering babies and infant children, in particular—but one which is also available to older children and adults as well.

It is an object of the invention, also, to provide such an RSV management and treatment technique which can be administered in a home environment, instead of to a hospital-type usage.

It is another object of the invention to provide this RSV management and treatment technique in a way as to be easily administrable by a parent or adult user, generally without any extensive training in the manner of its implementation.

And it is a further object of the invention to provide this management and treatment technique to deal with the effects of such other respiratory ailments as are attendant as coughs and colds.

### SUMMARY OF THE INVENTION

As will be seen from the following description, the RSV management and treatment technique of the invention follows from a recognition of the known benefits to be derived from the use of thermal packs from which heat energy is applied to specific areas of a user's body in providing consistent, convenient and comfortable heat application. The invention also follows from joining with the thermal advantage that produces, the use of topical ointments which provide medicated vapors which work to relieve a user's cough. Even though such ointments are not recommended for use with children under 2 years of age (where, for children over 2 years of age and adults, the directions for use typically call for rubbing a thick layer of the ointment on a chest or throat, for example, or for rubbing the ointment on sore, aching muscles), the present invention will be understood to encompass a cotton or other cloth impregnated with the ointment superimposed onto the thermal pack with the resultant combination then being applied as a single layer about the baby's or infant child's chest.

### BRIEF DESCRIPTION OF THE DRAWINGS

These and other features of the present invention will be more clearly understood from a consideration of the following description, taken in connection with the accompanying drawings in which:

FIG. 1 is a top plan view of a thermal wrap helpful in an understanding of the invention;

FIG. 2 illustrates a pattern for using an elastic thermal wrap according to the present invention for placement on the chest of a sitting or reclining RSV suffering baby or infant-child to enhance nasal inhalation; and

FIG. 3 is a side view of a warming vapor chest pad embodying the invention.

### DETAILED DESCRIPTION OF THE INVENTION

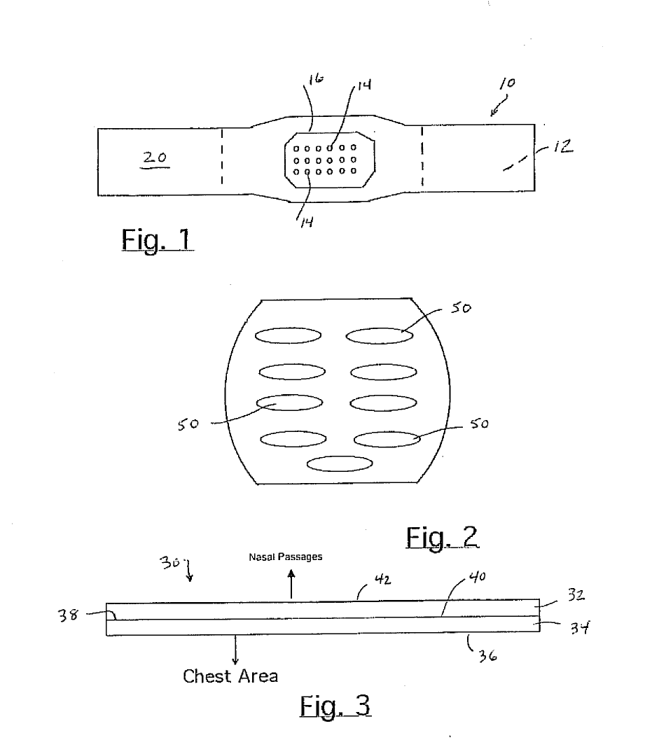

FIG. 1 is a top plan view of a thermal wrap as sold by the Proctor & Gamble Company of Cincinnati, Ohio. Such thermal wrap comprises one or more thermal packs having at least one continuous layer of a material which preferably exhibits specific thermal physical properties, and a plurality of individual heat cells which preferably comprise an exothermic composition, spaced apart and fixed within or to the structure of the thermal pack. Termed “disposable”, such thermal wraps may be stored in a substantially air-impermeable container and applied to a user's body as often as required for the relief of pain. After the heat source, i.e., the heat cell(s) or thermal pack(s), has been fully expended, the wrap is intended to be thrown away, as by being deposited in a suitable trash receptacle.

In FIG. 1, a flexible material 10 has an underside 12 and a plurality of heat cells 14 indicated within a pattern 16 on a top side 20. Typically, the dimensions of the pattern 16 are set out in accordance with the use to which the thermal pack is to be employed, as is its positioning within the wrap itself. In a typical manufacture, each thermal pack includes a plurality of these individual heat cells 14, embedded within a laminate structure of the thermal pack although each thermal pack may alternatively comprise a single continuous base layer in which individual, or groups of such heat cells are fixedly attached and spaced.

In use, the thermal wrap is placed with the top side 20 against the body part to be heated with the underside 12 facing “up”. Directions call for the frequent checking of the skin for irritation or burns developing from the exothermic composition, or for the imposition of a clothing layer between the skin and the heat cells, For those users 55 and older, the directions call for a “wearing” only while awake and not for sleeping. The uses are for providing temporary relief of minor muscular and joint aches and pains associated with overexertion, stress, sprains and arthritis.

An example of a different pattern for using a disposable thermal wrap as a warming vapor chest pad for enhanced nasal inhalation treatment of a user's cough, cold or respiratory infection is illustrated in FIG. 2. Such a pad is especially beneficial for an RSV suffering baby or infant-child according to the invention—and for older children and adults as well when employed in the manner illustrated by FIG. 3.

A mentholated topical cream ointment impregnated onto a cotton or like fabric according to the invention is utilized as an overlay atop the thermal wrap to not only serve to open the nasal airways of a wheezing child but so that the heat generated by the thermal wrap goes further in calming any spasms that might be present. The combination of the vapors together with the heat in a configuration suitable for a placement against the chest area serves the purpose—and in an arrangement light enough for simple packaging and shipping by a manufacturer. In this utilization, the vapors of the mentholated ointment significantly lessen the tendency for the baby or infant-child to have to breathe faster to bring the needed oxygen in and out of the lungs, which previously had been clogged because of the mucous present.

FIG. 3 illustrates a side view of the warming vapor pad 30 of the invention with a cotton or cloth fabric layer 32 impregnated with the topical ointment superimposed on an elastic thermal wrap 34. A downwardly facing first surface 36 of the thermal wrap 34 is adapted for placement against the chest of the sitting or reclining user; an upwardly facing second surface 38 of the wrap 34 then includes one or more heat cells of exothermic composition, preferably a plurality of them as in the arrangement of FIG. 2. A first surface 40 of the fabric layer 32 overlies the second surface 38 of the thermal wrap 34, while a second surface 42 of the fabric layer 32 faces upwardly, away from the chest area and towards the nasal passages. Contradistinct from the Proctor & Gamble utilization, the exothermic heat cells of the present invention are not in contact with the user's skin. Also differing from the prior Proctor & Gamble usage, the warming vapor pad for this nasal inhalation treatment is intended to just lie or rest against the chest area of the user, instead of being designed to encircle the torso with reclosable fastening means. And with the present invention, a further advantage follows from the heat cells being embedded within a laminate structure of the thermal wrap, fixed on its outer surface or included within pockets of the wrap, as at 50 in FIG. 2.

In a preferred construction of the invention, the mentholated ointment has as its active ingredients camphor, eucalyptol and menthol—with cedar leaf oil, nutmeg oil, turpentine oil, thymol and petrolatum as its inactive ingredients. One such cream ointment which provides positive results even when just coated over gauze layer materials atop the thermal wrap is available from the Proctor & Gamble Company as VICKS VAPORUB. There, camphor, eucalyptus oil and menthol are included as active ingredients to the extent 4.8%, 1.2% and 2.6% respectively.

Thus, with the invention, the exothermic cells heat the topical ointment impregnating the fabric layer 32 to provide a heated vapor flow upwardly through the opposing second surface 42 of the layer 32. Enhanced nasal inhalation then follows for the treatment of RSV suffering babies and infant children, as well as for older children and adults with coughs, colds or respiratory ailments and/or infections.

While there have been described what are considered to be preferred embodiments of the single integrated unit of the present invention, it will be appreciated that modifications can be made by those skilled in the art without departing from the scope of the teachings herein. For at least such reason, therefore, resort should be had to the claims appended hereto for a true understanding of the invention.

I claim:

1. A warming vapor pad for enhanced nasal inhalation comprising:
an elastic thermal wrap having a downwardly facing first surface adapted for placement against the chest of a sitting or reclining user, and an upwardly facing second surface including one or more heat cells of exothermic composition; and
a fabric impregnated with a topical ointment providing medicated vapors to relieve a user's cough, cold or respiratory infection;
with a first surface of the fabric superimposed on said second surface of said elastic thermal wrap directing a heated medicated vapor flow upwardly through an opposing second surface of said fabric.

2. The warming vapor pad of claim 1 wherein the elastic thermal wrap includes one or more thermal packs having a plurality of individual heat cells of exothermic composition.

3. The warming vapor pad of claim 2 wherein the elastic thermal wrap includes individual heat cells embedded within a laminate structure of the elastic thermal wrap.

4. The warming vapor pad of claim 2 wherein the elastic thermal wrap includes individual heat cells attached to a continuous base layer of the elastic thermal wrap.

5. The warming vapor pad of claim 2 wherein the elastic thermal wrap includes individual heat cells within spaced pockets about a continuous base layer of the elastic thermal wrap.

6. The warming vapor pad of claim 2 wherein the topical ointment is impregnated onto a cotton or cloth fabric as an overlay atop the elastic thermal wrap.

7. The warming vapor pad of claim 5 wherein the cotton or cloth fabric is impregnated with a mentholated topical cream.

8. The warming vapor pad of claim 7 wherein the mentholated topical cream includes camphor, eucalyptol and menthol as active ingredients.

9. The warming vapor pad of claim 7 wherein the mentholated topical cream includes cedar leaf oil, nutmeg oil, turpentine oil, thymol and petrolatum as inactive ingredients.

10. A warming vapor pad for enhanced nasal inhalation comprising:
an elastic thermal wrap having a downwardly facing first surface adapted for placement against the chest of a sitting or reclining user, and an upwardly facing second surface including one or more heat cells of exothermic composition; and
a fabric impregnated with a topical ointment providing medicated vapors to relieve a user's cough, cold or respiratory infection;
with a first surface of the fabric superimposed on said second surface of said elastic thermal wrap directing a heated medicated vapor flow upwardly through an opposing second surface of said fabric;
wherein the elastic thermal wrap includes one or more thermal wraps having a plurality of individual heat cells of exothermic composition;
wherein the topical ointment is impregnated onto a cotton or cloth fabric as an overlay atop the elastic thermal wrap;
wherein the cotton or cloth fabric is impregnated with a mentholated topical cream;
wherein the mentholated topical cream includes camphor, eucalyptol and menthol as active ingredients; and
wherein the mentholated topical cream includes cedar leaf oil, nutmeg oil, turpentine oil, thymol and petrolatum as inactive ingredients.
